(19) United States
(12) Patent Application Publication
(10) Pub. No.: US 2005/0208599 A1
(43) Pub. Date: Sep. 22, 2005

(54) Compositions and methods to select dendritic cells from a heterogeneous cell population

(75) Inventor: Ruth M. Ruprecht, Brookline, MA (US)

Correspondence Address: LAHIVE & COCKFIELD, LLP., 28 STATE STREET, BOSTON, MA, 02109, US

(73) Assignee: Dana-Farber Cancer Institute, Inc., Boston, MA (US)

(21) Appl. No.: 11/039,075

(22) Filed: Jan. 18, 2005

Related U.S. Application Data
(63) Continuation of application No. PCT/US03/22568, filed on Jul. 18, 2003.
(60) Provisional application No. 60/397,090, filed on Jul. 18, 2002.

(51) Int. Cl. G01N 33/567; C12N 5/08

(52) U.S. Cl. 435/7.21; 435/372

(57) ABSTRACT

The present invention is based, at least in part, on methods to select pure and functional dendritic cells from a heterogeneous population of cells. Accordingly, the invention features a method of selecting dendritic cells from a heterogeneous population of cells comprising contacting said cells with an antibody against dendritic cell-specific adhesion receptor (DC-SIGN), preferably DCN46, or a fragment thereof, and identifying dendritic cells based on binding of said antibody to said cells, thereby selecting dendritic cells from a heterogeneous population of cells.

### RELATED APPLICATIONS

This application is a continuation of PCT/U.S. 2003/022568, filed Jul. 18, 2003, which claims the benefit of U.S. Provisional Application Ser. No. 60/397,090, filed on Jul. 18, 2002, the entire contents of which are incorporated herein by this reference.

### GOVERNMENT FUNDING

Work described herein was supported, at least in part, by the National Institutes of Health (NIH) under grant R01 AI 43839, and by the Center for AIDS Research core grant IP3028691 awarded to Dana-Farber Cancer Institute. The U.S. government, therefore, may have certain rights in this invention.

### BACKGROUND OF THE INVENTION

The human DC-specific adhesion receptor DC-SIGN (CD209), a type II C-type lectin, facilitates the induction of primary immune responses (Geijtenbeek, T. B., et al. (2000) Cell 100:575; Bleijs, D. A., et al. (2001) Trends Immunol. 22:457) and plays a critical role during HIV infection (Geijtenbeek, T. B., et al. (2000) Cell 100:587; Pohlmann, S., et al. (2001) Trends Immunol. 22:643).

In vivo, DC-SIGN is expressed by immature dendritic cells (DC) in peripheral tissue as well as by mature DC in lymphoid tissue (Geijtenbeek, T. B., et al. (2000) Cell 100:575). Furthermore, DC-SIGN is expressed on two DC-precursor populations in peripheral blood (Geijtenbeek, T. B., et al. (2000) Nat. Immunol. 1:353). In addition, DC-SIGN expression has been detected on Hofbauer cells (Soilleux, E. J., et al. (2001) J Pathol. 195:586; Mummidi, S., et al. (2001) J. Biol. Chem. 276:33196). Alternative splicing events in DC-SIGN pre-mRNA generate a wide repertoire of DC-SIGN transcripts predicted to encode membrane-associated and soluble isoforms with varied binding domains and yet undiscovered biological properties (Mummidi, S., et al. (2001) J. Biol. Chem. 276:33196).

Monocytes and activated monocytes do not express DC-SIGN (Geijtenbeek, T. B., et al. (2000) Cell 100:575). However, DC-SIGN is rapidly upregulated during in vitro differentiation of monocytes into DC induced by the cytokines interleukin-4 (IL-4) and granulocyte-macrophage colony-stimulating factor (GM-CSF) (Geijtenbeek, T. B., et al. (2000) Cell 100:575). As binding and positive selection with monoclonal antibodies directed against cell surface molecules may alter cellular physiology (Dantal, J. and J. P. Soulillou (1991) Curr. Opin. Immunol. 3:740; Nizet, Y., et al. (1996) J. Immunol. Methods 199:1; Pierres, A., et al. (1992) Eur. J. Immunol. 22:413; Van Wauwe, J. P., et al. (1980) J. Immunol. 124:2708), a detailed analysis of DC function after treatment of DC with monoclonal antibody DCN46 directed against DC-SIGN was undertaken.

### SUMMARY OF THE INVENTION

The present invention is based, at least in part, on methods to select pure and functional dendritic cells from a heterogeneous population of cells. Accordingly, the invention features a method of selecting dendritic cells from a heterogeneous population of cells comprising contacting said cells with an antibody against dendritic cell-specific adhesion receptor (DC-SIGN), preferably DCN46, or a fragment thereof, and identifying dendritic cells based on binding of said antibody to said cells, thereby selecting dendritic cells from a heterogeneous population of cells. In one embodiment, the heterogeneous population of cells is sorted, e.g., via flow cytometry cell sorting, using magnetic beads, or by any other cell sorting method known in the art, following contacting the sample with the antibody.

In one aspect, a method of modulating an immune response in a subject, e.g., a mammal, preferably a human, is featured comprising the steps of obtaining a sample consisting of a heterogeneous population of cells; contacting said sample with an antibody against dendritic cell-specific adhesion receptor (DC-SIGN), preferably DCN46, or a fragment thereof; sorting said heterogeneous population of cells; identifying dendritic cells that bind to said antibody; contacting said dendritic cells with an antigen to produce antigenic cells; and administering said antigenic cells to said subject, thereby modulating an immune response in a subject. In one embodiment, the immune response is to a disease or disorder selected from the group consisting of viral infection, bacterial infection, parasitic infection, prion disease, neoplastic disease, allergy, and autoimmunity. In another embodiment, the antigenic cells are administered in a pharmaceutically acceptable formulation.

In another embodiment of the invention, a method of treating or preventing an immune disorder in a subject, e.g., mammal, preferably, a human, is featured, comprising the steps of obtaining a sample consisting of a heterogeneous population of cells; contacting said sample with an antibody against dendritic cell-specific adhesion receptor (DC-SIGN), preferably DCN46, or a fragment thereof; sorting said heterogeneous population of cells; identifying dendritic cells that bind to said antibody; contacting said dendritic cells with an antigen to produce antigenic cells; and administering said antigenic cells to said subject, thereby treating an immune disorder in a subject. In one embodiment, the immune disorder is selected from the group consisting of bacterial diseases, viral diseases, parasitic diseases, autoimmune diseases, allergy, and neoplastic diseases. In another embodiment, the antigenic cells are administered in a pharmaceutically acceptable formulation.

In one embodiment, the antigens used to contact said dendritic cells to produce an antigenic cell are selected from the group consisting of tumor cells, autoimmune cells, prions, bacterium, viruses, yeast and parasites, or, in another embodiment, are derived by recombinant means.

In a further embodiment, the present invention provides compositions for the treatment of immune diseases and disorders such as, for example, viral, bacterial and parasitic infections, prion diseases, prion diseases, autoimmune diseases and disorders, allergy and neoplastic diseases.

In another aspect, the invention features a method of producing a vaccine comprising the steps of obtaining a sample consisting of a heterogeneous population of cells; contacting said sample with an antibody against dendritic cell-specific adhesion receptor (DC-SIGN), preferably DCN46, or a fragment thereof; sorting said heterogeneous population of cells; identifying dendritic cells that bind to said antibody; and contacting said dendritic cells with an antigen, thereby producing a vaccine.

In a further embodiment, the heterologous cells used in the methods of the invention are derived from blood, lymph, lymph nodes, or spleen. In yet another embodiment, the dendritic cells identified by the methods of the invention are 99% pure, or preferably, 99.5% pure. In yet another embodiment, the antibody used in the kit of the invention may be labeled, e.g., fluorescently labeled or labeled using magnetic beads.

In still another aspect, the invention features a kit comprising an antibody against dendritic cell-specific adhesion receptor (DC-SIGN), preferably DCN46, or a fragment thereof; and instructions for use of said antibody to select dendritic cells from a heterogeneous population of cells. In one embodiment, the heterologous cells used in the kit of the invention are derived from blood, lymph, lymph nodes, or spleen. In another embodiment, the antibody used in the kit of the invention may be labeled, e.g., fluorescently labeled or labeled using magnetic beads.

### BRIEF DESCRIPTION OF THE DRAWINGS

FIGS. 1A-1B demonstrate DC-SIGN-based selection of DC by flow cytometry. (A) Typical scatterplot of DC culture on day 5. Large cells with high granularity (rectangle; DC) and contaminating lymphocytes (arrowhead) can be distinguished. (B) On day 5 of DC culture, cells were stained for DC-SIGN. The large cells identified in panel A (rectangle) were gated. Most of the cells within this gate stained positive for antibody DCN46 (bold line) when compared to isotype control (thin line) and were collected by flow cytometry.

FIG. 2 is a graph depicting that DC-SIGN antibody is not internalized into DC. DC were treated with (fixed) or without (unfixed) paraformaldehyde. Subsequently, DC were stained with unlabeled antibody DCN46 at 4° C. When DCN46-stained DC were incubated at 37° C., the cell surface-bound DC-SIGN antibody did not disappear from either fixed or unfixed DC as determined by immunostaining with a secondary antibody prior to FACS analysis. Mean and standard deviations of 3 experiments are shown.

FIG. 3 is a graph depicting the immunophenotype of DC-SIGN-sorted and unsorted immature DC. DC cultures were either unsorted or sorted for antibody DCN46 positive DC. Subsequently, the in vitro generated immature DC were stained with antibodies to the surface antigens indicated and analyzed by FACS. Mean and standard deviations of 4 experiments are shown.

FIGS. 4A-4B depict that binding of antibody DCN46 and subsequent flow cytometric sorting of DC cultures for DC-SIGN when compared to unsorted DC cultures did not alter the characteristic functions of DC such as the uptake of antigen (A) and the stimulation of a recall antigen response (B). (A) DC were incubated with red fluorescent latex beads at 37° C. and analyzed after varying lengths of time by FACS. Background due to nonspecific binding of latex beads to DC was determined by incubating sodium azide-treated DC with latex beads. This value was subtracted from the data shown. Mean and standard deviations of 3 experiments are shown. (B) The presentation of the recall antigen tetanus toxoid to PBL by DC was assessed by incubating sorted or unsorted DC and PBL at a ratio of 1:40 in the presence of different concentrations of tetanus toxoid for 3 days. T-cell proliferation was subsequently determined by [³H]-thymidine incorporation. Mean and standard deviations of 2 experiments are shown.

FIGS. 5A-5B depict DC-SIGN-based cell sorting does not affect the differentiation of immature DC. (A) The ability of unsorted and DC-SIGN-sorted immature DC (filled histogram) to differentiate into mature DC (bold lined histogram) upon LPS treatment was investigated by direct immunofluorescence. Isotype control antibody (dotted lined histogram) was used as negative control. The results from one representative experiment of four are shown. (B) To normalize data from experiments with DC from different donors, the mean fluorescence intensity of HLA-DR on DC-SIGN-sorted and matured DC was arbitrarily set to 100 in each set of experiment. Mean and standard deviations of 4 experiments are shown.

FIG. 6 is a graph depicting allogeneic DC induced T-cell proliferation is not affected by DC-SIGN-based cell sorting of DC. Mature DC and allogeneic PBL at various ratios were incubated for 5 days at 37° C. T-cell proliferation was subsequently determined by [³H]-thymidine incorporation. Mean and standard deviations of one representative experiment out of 4 run in duplicate are shown.

### DETAILED DESCRIPTION OF THE INVENTION

The present invention pertains to methods to select, e.g., positively select, pure and functional dendritic cells from a heterogeneous population of cells. “Dendritic cells” or “DC” are the sentinels of the immune system and as such are the first cells to come into contact with invading pathogens. They are professional antigen-presenting cells that efficiently capture antigens in the peripheral tissues and process these antigens to form MHC-peptide complexes. After antigen uptake, these immature DC acquire the unique capacity to migrate from the periphery to the T cell areas of secondary lymphoid organs, e.g., lymph nodes and spleen. As the cells travel, they mature and alter their profile of cell surface molecules to attract resting T cells, present their antigenic load and induce an immune response, e.g., T cell proliferation (Shaw, S., et al. (1986) Nature 323:262; Adema, G. J., et al. (1997) Nature 387:713; Banchereau, J. and Steinman, R. M. (1986) Nature 392:245).

As used herein, the term “immune cell” includes cells that are of hematopoietic origin and that play a role in the immune response. Immune cells include lymphocytes, such as B cells and T cells; natural killer cells; and myeloid cells, such as monocytes, macrophages, eosinophils, mast cells, basophils, and granulocytes.

As used herein, the term “T cell” refers to T lymphocytes as defined in the art and is intended to include thymocytes, immature T lymphocytes, mature T lymphocytes, resting T lymphocytes, or activated T lymphocytes. The T cells can be CD4+ T cells, CD8+ T cells, CD4+ CD8+T cells, or CD4−CD8− T cells. The T cells can also be T helper cells, such as T helper 1 (Th1) or T helper 2 (Th2) cells. The term T cells also includes activated T cells and memory T cells.

As used herein, the term “immune response” includes T cell mediated and/or B cell mediated immune responses that are influenced by modulation of T cell costimulation. Exemplary immune responses include T cell responses, e.g., cytokine production, and cellular cytotoxicity. In addition, the term immune response includes immune responses that are indirectly affected by T cell activation, e.g., antibody production (humoral responses) and activation of cytokine responsive cells, e.g., macrophages.

As used herein, an “antigen” is any substance that, as a result of coming in contact with appropriate cells, is capable of inducing a state of sensitivity and/or a specific immune response and reacts with the products of that response, that is, with specific antibodies or specifically sensitized T cells, or both. Antigens may be soluble substances, such as toxins and proteins, or particulates, such as bacteria, viruses and tissue cells. Antigens may be foreign or self-derived. Only the portion of the protein or polysaccharide molecule known as the antigenic determinant (epitopes) combines with antibody or a specific receptor on a lymphocyte.

“Positive selection” as used herein, indicates that DC cells are specifically targeted by a cell specific monoclonal antibody and selected from a heterogeneous population of cells. This is in contrast to cells that remain after depletion.

As used herein, the term “contacted with” includes exposure to, e.g., the exposure of cells to antibody or antigen.

As used herein, a “heterogeneous population of cells” refers to the mixture of cell types from a tissue, e.g., lymph nodes, spleen and blood, or a fluid, e.g., cerebrospinal fluid, lymph, serum, and plasma, or other tissues or fluids that contain dendritic cells. For example, blood may be described as being composed or serum and plasma and may contain red blood cells, monocytes, macrophages, B cells, T cells, etc. and lymph nodes may contain, epithelial cells, connective tissue cells, blood. The heterogeneous population of cells may be derived from a mammal, preferably a human. In another embodiment, the antibody is labeled by magnetic beads.

Migration of DC is essential to the generation of immune response since DC have to travel from their “nursery” to the inflamed tissue where they sample antigen and subsequently to the lymph nodes, where T cells reside. Upon arrival of DC in the lymph node, DC-T cell clustering enables T cells to scan the peptides presented by DC, and the recognition of specific peptides triggers the activation of T cells. These primed T cells migrate in the blood to the site of infection and/or inflammation where they perform their immune function (Sallusto, F. and Lanzacecchia, A. (1999) J. Exp. Med. 189:611).

DC-specific ICAM-3 grabbing nonintegrin (DC-SIGN), also known as CD209, is a DC specific receptor that mediates strong adhesion between DC and ICAM-3 on resting T cells and is essential for DC migration and DC-induced T cell proliferation (Gejitenbeek, T. B., et al. (2000) Cell 100:575). DC-SIGN is a cell adhesion receptor with a DC restricted expression pattern (Gejitenbeek, T. B., et al. (2000) Cell 100:575; Gejitenbeek, T. B. H., et al. (2000) Cell 100:587). In vivo, DC-SIGN is expressed by immature DC in the peripheral tissue as well as mature DC in the lymphoid tissues such as lymph nodes, tonsils and spleen. In skin, DC-SIGN expression is found on dermal DC but not CD1a+ Langerhans cells in the epidermis. In mucosal tissues, DC-SIGN is expressed in the rectum, uterus, cervix and lamina propria, substantiating the importance of the localization of DC as a first-line of defense against invading pathogens and viruses (Gejitenbeek, T. B., et al. (2000) Cell 100:575). Two DC-SIGN+ DC-precursor populations that differ in the expression of CD14 were found in peripheral blood (Gejitenbeek, T. B., et al. (2000) Nat Immunol 1:353). The in vitro generation of DC demonstrates that DC-SIGN is up-regulated rapidly on monocytes upon differentiation induced by the cytokines interleukin-4 (IL-4) and granulocyte-macrophage colony-stimulating factor (GM-CSF). Further in vitro maturation of DC with tumor necrosis factor α (TNFα) reduces the expression of DC-SIGN to some extent (Gejitenbeek, T. B., et al. (2000) Cell 100:575).

DC-SIGN is a type II transmembrane protein, containing a mannose-binding C-type lectin domain that forms the ligand-binding site (Gejitenbeek, T. B., et al. (2000) Cell 100:575; Curtis, B. M., et al. (1992) Proc. Natl. Acad. Sci., USA 89:8356). The binding of ligand to DC-SIGN is activation-independent, but dependent on the binding of two Ca+2 ions to the carbohydrate recognition domain (CRD). One of the bound Ca+2 ions forms the core of the sugar-binding site and coordinates the binding of ligand directly, whereas the second is required to stabilize the binding pocket (Drickamer, K. (1999) Immunol. Today 20:240).

The natural ability of DC to migrate, their potent antigen presenting ability and their ability to initiate T-cell mediated immune responses indicate that DC play a vital role in the immune system. Indeed, DC activate T cells more efficiently than any other known antigen presenting cell, and are required for the initial activation of naive T cells in vitro and in vivo.

In addition to naive T cell activation, DC can influence the balance of the Th1/Th2 immune response. Several reports have indicated that DC preferentially activate Th1 responses, with the major determining factor being IL-12 secretion from the activated DC (Macatonia, et al. (1995) J. Immunol. 154:5071; Hilkens, et al. (1997) Blood 90:1920). Other reports have shown that DC can induce the generation of either Th1 or Th2 clones (Roth, et al. (1996) Scand. J. Immunol. 43:646). The pivotal role played by DC in antigen presentation and T cell activation has resulted in considerable interest in the use of DC in immunotherapy, e.g., as cellular adjuvants or as cell based vaccines, particularly in the areas of vaccinology and immunotherapy, especially cancer immunotherapy. See, e.g., WO98/23728 and U.S. Pat. No. 6,503,503, Bigner, et al. Furthermore, DC-SIGN was originally identified by cloning of a cDNA from a placental library based on its ability to bind to the HIV-1 envelope glycoprotein, gp120 (Curtis, B. M., et al. (1992) Proc. Natl. Acad. Sci., USA 89:8356). Subsequently, it has been shown that DC-SIGN promotes efficient infection of CD4+ T cells in trans by exploiting the machinery of DC and the properties of DC-SIGN. DC-SIGN binds to HIV-1 with high affinity (exceeding that of CD4), sequesters it, thus facilitating its transport from the periphery to secondary lymphoid tissues rich in T cells, which then allows an enhanced infection of HIV-1 target cells, all through use of the natural migratory function of DC (Gejitenbeek, T. B., et al. (2000) Cell 100:587).

Thus, it is desirable to have pure and functional DC. Methods known in the art for isolating DC from heterogeneous populations of cells, e.g., peripheral blood mononuclear cells (PBMC), rely on depletion of cell types other than DC, e.g., immunomagnetic depletion or through the use of monoclonal antibody cocktails, to isolate DC. Examples of available methods and kits utilizing depletion methods to isolate DC can be found at and purchased from Miltenyi Biotec Inc., 12740 Earhart Avenue, Auburn, Calif., and Stem Cell Technologies, Inc., Vancouver, British Columbia, Canada. These methods produce DC, however, the purity of sample is often wanting. As an alternative to depletion isolation of DC, positive selection of DC may be used. However, positive selection of DC with monoclonal antibodies directed against cell surface molecules often alters the cellular physiology of the cells so that they no longer display their normal characteristics (Dantal, J. and J. P. Soulillou (1991) Curr. Opin. Immunol. 3:740; Nizet, Y., et al. (1996) J. Immunol. Methods 199:1; Pierres, A., et al. (1992) Eur. J. Immunol. 22:413; Van Wauwe, J. P., et al. (1980) J. Immunol. 124:2708), e.g., uptake, processing, presentation of antigen to T cells and induction of an immune response. Accordingly, the present invention features a method of selecting dendritic cells from a heterogeneous population of cells comprising the step of contacting said cells with an antibody against dendritic cell-specific adhesion receptor, DC-SIGN, preferably DCN46, or a fragment thereof, i.e., a functional fragment, capable of binding to DC-SIGN. In one embodiment, cells contacted by the antibody can be sorted using flow cytometry cell sorting, also referred to as Fluorescence-activated Cell Sorting (FACS), a technique known in the art for separation, classification and quantitation of cells (Orfao, A, and Ruiz-Arguelles, A. (1996) Clin. Biochem. 29:5). In another embodiment, cells contacted by the antibody can be sorted using magnetic beads, e.g., immunomagnetic beads (Rubbi, C. P., and Rickwood, D. (1996) J. Immunol. Methods 192:157). The DC isolated using the methods of the invention are both pure and functional.

The present invention provides a method for selecting of DC that are at least 90% pure, preferably, at least 91%, 92%, 93%, 94%, 95%, 95.5%, 96%, 96.5%, 97%, 97.5%, 98%, 98.5%, 99%, 99.5%, 99.8%, 99.9% pure. Methods known in the art for assessing the purity and functionality of DC include but are not limited to, expression of CD40, CD80, CD83 and CD86, as well as HLA-DR, phagocytosis of large particles, antigen uptake, stimulation of recall antigen response and T cell proliferation. See, Hart, D. (1997) J. Am. Sco. Hematol. 90:3245, incorporated herein by reference.

The present invention also features a kit comprising an antibody that selects pure and functional dendritic cells from a heterogeneous population of cells comprising a dendritic cell specific antibody and instructions for use of said antibody. In one embodiment, the antibody is DCN46, or a fragment thereof. The kit preferably comprises a box or container that holds the components of the kit. The box or container is affixed with a label or a Food and Drug Administration approved protocol. The box or container holds components of the invention that are preferably contained within plastic, polyethylene, polypropylene, ethylene, or propylene vessels. The vessels can be capped-tubes or bottles.

Antibodies of the Invention

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “monoclonal antibody” as used herein, refers to a population of antibody molecules that contain only one species of an antigen binding site. A monoclonal antibody composition thus typically displays a single binding affinity for a particular protein with which it immunoreacts. In one embodiment of the invention, the antibody is fluorescently labeled.

The term “antigen-binding portion” of an antibody (or simply “antibody portion”), as used herein, refers to one or more fragments of an antibody that retain the ability to specifically bind to an antigen (e.g., DC-SIGN). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423; and Huston et al. (1988) Proc. Natl. Acad. Sci., USA 85:5879). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci., USA 90:6444; Poljak, R. J., et al. (1994) Structure 2:1121). Binding fragments are produced by recombinant DNA techniques, or by enzymatic or chemical cleavage of intact immunoglobulins. Binding fragments include Fab, Fab′, F(ab′)₂, Fabc, Fv, single chains, and single-chain antibodies. Other than “bispecific” or “bifunctional” immunoglobulins or antibodies, an immunoglobulin or antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann (1990) Clin. Exp. Immunol. 79:315; Kostelny, et al. (1992) J. Immunol. 148:1553.

Methods of Treatment

The present invention provides methods for modulating an immune response, e.g., an immune disease or disorder, such as, and without limitation, infectious diseases (bacterial, viral, and parasitic), prion diseases, autoimmune diseases, allergy, and neoplastic diseases, in subjects (e.g., mammals, such as humans). In one aspect, the invention pertains to a method of modulating an immune disease or disorder which can be treated or prevented by modulating an immune response, e.g. through the induction of an appropriate immune response, comprising the steps of, obtaining a sample consisting of a heterogeneous population of cells, contacting said sample with an antibody against dendritic cell-specific adhesion receptor, DC-SIGN, sorting said heterogeneous population of cells by flow cytometry cell sorting, identifying DC cells that bind to said antibody, contacting said DC cells with antigen, and administering said antigenic cells to a patient, thereby provoking an immune response. For example, the pure and functional DC of the invention can be contacted by to tumor derived antigens and then can be administered to a patient, thereby provoking an anti-tumor immune response in the patient. Similarly, infectious diseases can be treated by administering to the patient the pure and functional DC of the invention once exposed to antigens derived from the infectious agent.

Another aspect of the present invention is the treatment of immune diseases and disorders in a subject, preferably a mammal, more preferably a human, comprising the steps of, obtaining a sample consisting of a heterogeneous population of cells, contacting the sample with an antibody against dendritic cell-specific adhesion receptor (DC-SIGN), sorting the heterogeneous population of cells, identifying dendritic cells that bind to the antibody, contacting the isolated dendritic cells with an antigen capable of inducing an immune response, and administering the antigenic cells to the subject, thereby treating an immune disease and/or disorder.

The term “treatment” or “treating” as used herein refers to either (1) the prevention of a disease or disorder (prophylaxis), or (2) the reduction or elimination of symptoms of the disease or disorder (therapy). The terms “prevention”, “prevent” or “preventing” as used herein refers to inhibiting, averting or obviating the onset or progression of a disease or disorder (prophylaxis).

The DC isolated by the methods of the invention can also be used in a number of other immunotherapies such as ex vivo cell transplantation therapies for treating diseases and disorders of the immune system, such as AIDS; the ex vivo expansion of T cells, particularly antigen specific T cells which can then be used to treat diseases and disorders characterized by deterioration of the immune system; the preparation of antigen activated DC according to methods known in the art; and development of vaccines and vaccine adjuvants.

The antigen may be any antigen against which the mammal is capable of mounting an immune response. The antigen may be protein, carbohydrate or nucleic acid in nature and may be derived from any suitable source, including neoplastic cells (e.g., tumor cells), prions, autoimmune cells, and infectious agents (e.g., bacterium, virus, yeast, parasite). Alternatively, the antigen can be derived by recombinant means.

Recombinant nucleic acids encoding antigens may be isolated and purified free from other nucleotide sequences by ordinary purification techniques, e.g., using restriction enzymes to isolate desired fragments. The nucleic acid may also be synthesized in vitro, using standard methodology. A recombinant nucleic acid according to the invention includes nucleic acid molecules comprised of DNA or RNA, including coding and regulatory sequences, as well as vector sequences. Recombinant nucleic acids are molecules which are not found in nature. They have been engineered to join together originally separate sequences, usually from different chromosomes or organisms.

Any of the techniques which are available in the art may be used to introduce the recombinant nucleic acid encoding the desired antigen into the dendritic cell. These techniques are collectively referred to as transfection herein. The terms “transfection” or “transfected with” refers to the introduction of exogenous nucleic acid into a mammalian cell and encompass a variety of techniques useful for introduction of nucleic acids into mammalian cells including electroporation, calcium-phosphate precipitation, DEAE-dextran treatment, lipofection, microinjection and infection with viral vectors, e.g., viral vectors. Suitable methods for transfecting mammalian cells can be found in Sambrook et al. (Molecular Cloning: A Laboratory Manual, 2nd Edition, Cold Spring Harbor Laboratory press (1989)) and other laboratory textbooks. Choice of suitable vectors for expression is well within the skill of the art. The nucleic acid is “in a form suitable for expression” in which the nucleic acid contains all of the coding and regulatory sequences required for transcription and translation of a gene, which may include promoters, enhancers and polyadenylation signals, and sequences necessary for transport of the molecule to the surface of the tumor cell, including N-terminal signal sequences. When the nucleic acid is a cDNA in a recombinant expression vector, the regulatory functions responsible for transcription and/or translation of the cDNA are often provided by viral sequences. Examples of commonly used viral promoters include those derived from polyoma, Adenovirus 2, cytomegalovirus and Simian Virus 40, and retroviral LTRs. Regulatory sequences linked to the cDNA can be selected to provide constitutive or inducible transcription, by, for example, use of an inducible promoter, such as the metallothienin promoter or a glucocorticoid-responsive promoter.

A preferred approach for introducing nucleic acid into tumor cells is by use of a viral vector containing nucleic acid. Examples of viral vectors which can be used include retroviral vectors (Eglitis, M. A., et al. (1985) Science 230:1395; Danos, O. and Mulligan, R. (1988) Proc. Natl. Acad. Sci., USA 85:6460); Markowitz, D., et al. (1988) J. Virol. 6:1120), adenoviral vectors (Rosenfeld, M. A., et al. (1992) Cell 68:143) and adeno-associated viral vectors (Tratschin, J. D., et al. (1985) Mol. Cell. Biol. 5:3251). Infection of tumor cells with a viral vector has the advantage that a large proportion of cells will receive nucleic acid, thereby obviating a need for selection of cells which have received nucleic acid, and molecules encoded within the viral vector, e.g. by a cDNA contained in the viral vector, are expressed efficiently in cells which have taken up viral vector nucleic acid.

Alternatively, nucleic acids can be expressed on a tumor cell using a plasmid expression vector which contains nucleic acid. Suitable plasmid expression vectors include CDM8 (Seed, B. (1987) Nature 329:840) and pMT2PC (Kaufman, I. (1987) EMBO J. 6:187). Since only a small fraction of cells (about 1 out of 105) typically integrate transfected plasmid DNA into their genomes, it is advantageous to transfect a nucleic acid encoding a selectable marker into the tumor cell along with the nucleic acid(s) of interest. Preferred selectable markers include those which confer resistance to drugs such as G418, hygromycin and methotrexate. Selectable markers may be introduced on the same plasmid as the nucleic acid(s) of interest or may be introduced on a separate plasmid.

A homogenous population of transfected tumor cells can be prepared by isolating a single transfected cell by limiting dilution cloning followed by expansion of the single cell into a clonal population of cells by standard techniques. Antigen expression may be determined by any of a variety of methods known in the art, such as immunocytochemistry, ELISA, Western blotting, radioimmunoassay, or protein fingerprinting.

As used herein, the term “immune disorder” includes any disease, disorder or condition that can be treated or prevented through the modulation, e.g., upregulation or down-regulation, of an immune response. As used herein, the term “modulating” means changing or altering, and embraces both upmodulating and downmodulating.

The methods of the present invention are effective for preventing, treating or eliminating disease caused by a variety of viruses. As used herein, the term “viral infection” includes infections with organisms including, but not limited to, HIV (e.g., HIV-1 and HIV-2), human herpes viruses, cytomegalovirus (esp. Human), Rotavirus, Epstein-Barr virus, Varicella Zoster Virus, hepatitis viruses, such as hepatitis B virus, hepatitis A virus, hepatitis C virus and hepatitis E virus, paramyxoviruses: Respiratory Syncytial virus, parainfluenza virus, measles virus, mumps virus, human papilloma viruses (for example HPV6, 11, 16, 18 and the like), flaviviruses (e.g. Yellow Fever Virus, Dengue Virus, Tick-borne encephalitis virus, Japanese Encephalitis Virus) or influenza virus.

The methods of the present invention are effective for preventing, treating or eliminating disease caused by a variety of bacterial organisms. As used herein, the term “bacterial infections” include infections with a variety of bacterial organisms, including gram-positive and gram-negative bacteria. Examples include, but are not limited to, Neisseria spp, including N. gonorrhea and N. meningitidis, Streptococcus spp, including S. pneumoniae, S. pyogenes, S. agalactiae, S. mutans; Haemophilus spp, including H. influenzae type B, non typeable H. influenzae, H. ducreyi; Moraxella spp, including M catarrhalis, also known as Branham ella catarrhalis; Bordetella spp, including B. pertussis, B. parapertussis and B. bronchiseptica; Mycobacterium spp., including M. tuberculosis, M. bovis, M. leprae, M. avium, M paratuberculosis, M. smegmatis; Legionella spp, including L. pneumophila; Escherichia spp, including enterotoxic E. coli, enterohemorragic E. coli, enteropathogenic E. coli; Vibrio spp, including V. cholera, Shigella spp, including S. sonnei, S. dysenteriae, S. flexnerii; Yersinia spp, including Y enterocolitica, Y pestis, Y. pseudotuberculosis, Campylobacter spp, including C. jejuni and C. coli; Salmonella spp, including S. typhi, S. paratyphi, S. choleraesuis, S. enteritidis; Listeria spp., including L. monocytogenes; Helicobacter spp, including H. pylori; Pseudomonas spp, including P. aeruginosa, Staphylococcus spp., including S. aureus, S. epidermidis; Enterococcus spp., including E. faecalis, E. faecium; Clostridium spp., including C. tetani, C. botulinum, C. difficile; Bacillus spp., including B. anthracis; Corynebacterium spp., including C diphtheriae; Borrelia spp., including B. burgdorferi, B. garinii, B. afzelii, B. andersonii, B. hermsii; Ehrlichia spp., including E. equi and the agent of the Human Granulocytic Ehrlichiosis; Rickettsia spp, including R. rickettsii; Chlamydia spp., including C trachomatis, C. neumoniae, C. psittaci; Leptsira spp., including L. interrogans; Treponema spp., including T. pallidum, T. denticola, T. hyodysenteriae.

The methods of the present invention are effective for preventing, treating or eliminating disease caused by a variety of protozoal and parasitic organisms. As used herein, the term “parasitic infection” include infections with a variety of parasitic organisms, including, but not limited to Anaplasma, Babesia, Balantidium, Besnoitia, Chlamydia, Coccidia, Cryptosporondium, Cytauxzoon, Eimeria Entamoeba, Eperythrozoon, Erlichia, Giardia, Haemobartonella, Hammondia, Isopora, Leishmania, Neorickettsia, Plasmodium, Pneumocystis, Rickettsia, Schistosoma, Sarcocystis, Theileria, Thrichinella, Toxoplasma, Trichomonas, Trypanosoma, Unicaria, Dipylidium, Echinococcuse, Taenia, Ancylostoma, Ascaris, Enterobius, Strongyloides, Strongylus, Toxocara, Toxascaris and Trichuris.

The methods of the present invention are effective for preventing, treating or eliminating disease caused by prions, such as, but not limited to, familial Creutzfeldt-Jakob disease, Gerstmann-Straussler-Scheinker disease, bovine spongiform encephalopathy (BSE), scrapie and fatal familial Insomnia. As used herein, the term “prion” or “prion disease” refers to a group of transmissible spongiform encephalopathies or TSE. TSEs are caused by abnormalities of the prion protein (PrP). For example, Creutzfeldt-Jakob disease is caused by the conversion of the normal protease-sensitive PrP iso form, designated PrP(C), to a protease resistant isoform, designated PrP(Sc). The change of PrPC into PrPSc can occur spontaneously, however, it can also be induced by PrPSc. PrP(Sc) forms into an infectious particle, named a ‘prion’ that can transmit the disease. The process by which prions proceed to the central nervous system (CNS) following peripheral uptake is referred to as neuroinvasion Accumulation of PrP(Sc) in the brain causes degenerative disorders affecting the CNS leading to neurodegeneration.

As used herein, the term “autoimmunity” or “autoimmune disease or disorder” refers to the condition in which a subject's immune system starts reacting against his or her own tissues. Non-limiting examples of autoimmune diseases and disorders having an autoimmune component that may be treated according to the invention include type 1 diabetes, arthritis (including rheumatoid arthritis, juvenile rheumatoid arthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia greata, allergic responses due to arthropod bite reactions, Crohn's disease, iritis, conjunctivitis, keratoconjunctivitis, ulcerative colitis, asthma, allergic asthma, cutaneous lupus erythematosus, scleroderma, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

As used herein, the term “allergy” refers to the state of hypersensitivity induced by exposure to a particular antigen (allergen) that results in harmful immunologic reactions on subsequent exposures. Examples of allergens include, but are not limited to, plant, animal, bacterial, and parasitic allergans as well as metal-based allergens that cause contact sensitivity. Other allergans include weed, grass, tree, peanut, mite, flea and cat antigens.

As used herein, the term “neoplastic disease” is characterized by malignant tumor growth or in disease states characterized by benign hyperproliferative and hyperplastic cells. The common medical meaning of the term “neoplasia” refers to “new cell growth” that results as a loss of responsiveness to normal growth controls, e.g., neoplastic cell growth.

As used herein, the terms “hyperproliferative”, “hyperplastic”, “malignant” and “neoplastic” are used interchangeably, and refer to those cells in an abnormal state or condition characterized by rapid proliferation or neoplasia. The terms are meant to include all types of hyperproliferative growth, hyperplastic growth, cancerous growths or oncogenic processes, metastatic tissues or malignantly transformed cells, tissues, or organs, irrespective of histopathologic type or stage of invasiveness. A “hyperplasia” refers to cells undergoing an abnormally high rate of growth. However, as used herein, the terms neoplasia and hyperplasia can be used interchangeably, as their context will reveal, referring generally to cells experiencing abnormal cell growth rates. Neoplasias and hyperplasias include “tumors,” which may be either benign, premalignant or malignant.

The terms “neoplasia,” “hyperplasia,” and “tumor” are often commonly referred to as “cancer,” which is a general name for more than 100 diseases that are characterized by uncontrolled, abnormal growth of cells. Examples of cancer include, but are not limited to: breast; colon; non-small cell lung, head and neck; colorectal; lung; prostate; ovary; renal; melanoma; and gastrointestinal (e.g., pancreatic and stomach) cancer; and osteogenic sarcoma.

The term “tumor antigen” as used herein relates to any antigen expressed on a tumor cell, including but not limited to, Mucin1, carcinoembryonic antigen, oncofetal antigens and tumor-associated antigens. Also included in this definition are any antigens expressed by tumor cells that are encoded by a single DNA strand.

As used herein, the term “subject” is intended to include all vertebrates, i.e. human and non-human animals. The term “non-human animals” of the invention includes, but is not limited to, mammals, rodents, mice, and non-mammals, such as non-human primates, sheep, dog, horse, cow, chickens, amphibians, reptiles and the like. In one embodiment, the subject is a mammal, e.g., a human.

Use of DC as Vaccines

The present invention provides methods for producing a vaccine comprising the steps of, obtaining a sample consisting of a heterogeneous population of cells; contacting the sample with an antibody against dendritic cell-specific adhesion receptor (DC-SIGN); sorting the heterogeneous population of cells; identifying dendritic cells that bind to the antibody; and contacting the isolated dendritic cells with an antigen capable of inducing an immune response.

For example, tumor-associated antigens can be prepared from tumor cells, either by preparing crude lysates of tumor cells (Cohen et al. (1994) Cancer Res. 54:1055; Cohen et al. (1994) Eur. J. Immunol. 24:315), or by partially purifying the antigens (Itoh et al. (1994) J. Immunol. 153:1202). Moreover, useful tumor antigens may be purified further, or even expressed recombinantly, to provide suitable antigen preparations. Vaccines produced in this manner can also help reduce the risks of conditions such as experimental allergic encephalitis and other auto-immune complications (Strauss et al. (1982); Dal Canto et al. (1995); Swanborg (1995)).

Purified dendritic cells are contacted with (e.g., exposed to) antigen, to allow them to take up the antigen in a manner suitable for presentation to other cells of the immune system. Numerous methods of contacting dendritic cells with antigen are known in the art. those of skill in the art regard development of suitable methods for a selected antigen as routine experimentation. In general, the antigen is added to cultured dendritic cells under conditions promoting viability of the cells, and the cells are then allowed sufficient time to take up and process the antigen, and express antigen peptides on the cell surface in association with either Class I or Class II MHC, a period of about 24 hours (from about 18 to about 30 hours, preferably, about 24 hours). Dendritic cells may also be exposed to antigen by transfecting them with DNA encoding the antigen.

A “vaccine”, as used herein, is a preparation which contains a dendritic cell that expresses a specific antigen and has the ability to induce an immune response directed against the antigen. The vaccine of the present invention can be used for therapeutic methods of treating, preventing or ameliorating a subject at risk for or having an immune disease or disorder. The vaccine of the present invention can be incorporated into pharmaceutical compositions suitable for administration. Such compositions typically comprise antigenic dendritic cell and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Pharmaceutical Compositions

A “pharmaceutically acceptable formulation” of the invention is formulated to be compatible with its intended route of administration. The term “administration” or “administering” is intended to include routes of introducing the antigenic dendritic cells to a subject to perform their intended function. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutically acceptable formulations suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

### EXAMPLES

This invention is further illustrated by the following examples which should not be construed as limiting. The contents of all references, figures, patents and published patent applications cited throughout this application are hereby incorporated by reference.

A. Materials and Methods

Reagents

The following cell culture supplements were used: granulocyte-macrophage colony-stimulating factor (GM-CSF) (Leukine; Immunex, Seattle, Wash.), interleukin-4 (IL-4) (PeproTech, Rocky Hill, N.J.) and lipopolysaccharide (LPS; Sigma, Saint Louis, Mo.). Tetanus toxoid from Clostridium tetani was purchased from Calbiochem (La Jolla, Calif.).

Generation and Flow Cytometric Selection of Monocyte-Derived DC

DC were generated from buffy coats of anonymous healthy donors (provided by Children's Hospital, Boston, Mass.) as described recently (Gruber, A., et al. (2000) Blood 96:1327). Briefly, peripheral blood mononuclear cells (PBMC) were isolated by density centrifugation in Ficoll-Paque (Pharmacia, Uppsala, Sweden). Plastic-adherent PBMC were incubated for 7 days in RPMI 1640 medium (Gibco BRL, Gaithersburg, Md.) supplemented with 10% heat-inactivated human serum AB (Sigma), GM-CSF (20 ng/ml) and IL-4 (20 ng/ml) in order to generate immature DC. On day 5 of culture, cells were stained with FITC-conjugated anti-DC-SIGN (clone DCN46; Becton Dickinson, Mountain View, Calif.) and subsequently, positively stained DC were selected using the MoFlow cell sorter (Cytomation, Fort Collins, Colo.). The sorted DC were incubated for 2 more days in culture medium supplemented with GM-CSF and IL-4 and finally matured by addition of 10 ng/ml of LPS to the culture medium.

Immunofluorescence

Immunophenotyping of cells was accomplished by using phycoerythrin (PE)-conjugated anti-CD3, anti-CD11c, anti-CD14, anti-CD19, anti-CD56, anti-CD80, anti-CD86, anti-DC-SIGN, anti-HLA-DR, isotype control antibody (all from Becton Dickinson), anti-CD40 (Biosource Intl., Camarillo, Calif.) and anti-CD83 (Immunotech, Marseille, France). The analyses were carried out on a flow cytometer (Coulter Epics; Beckman Coulter, Miami, Fla.).

Internalization Assay

Cell-surface bound antibody internalization by DC was measured as described (Cella, M., et al. (1997) J. Exp. Med. 185:1743). Immature DC, treated with (fixed) or without (unfixed) 1% (v/v) paraformaldehyde, were stained with unlabeled anti-DC-SIGN (clone DCN46; Becton Dickinson) at 4° C. Subsequently, DC were incubated at 37° C. to allow internalization. At various time points, DC samples were taken and stained with a PE-labeled secondary antibody at 4° C. The fluorescence intensity of the stained DC was measured by FACS. The amount of internalization for DC incubated at 37° C. was determined by the percentage decrease of median fluorescence intensity as compared to fixed control samples.

Phagocytosis of Latex Beads

Immature DC (10⁵ DC in 500 μl medium) were coincubated with 5×10⁶ red fluorescent microspheres (latex, diameter 1 μm; Sigma) for varying periods of time. To distinguish nonspecifically bound beads from phagocytosed beads, the cells were poisoned with 1.0% (w/v) sodium azide before addition of red fluorescent microspheres. At the end of the assay, cells were separated from unengulfed beads by density gradient centrifugation and analyzed by FACS as described (Gruber, A., et al. (2000) Blood 96:1327).

Autologous Mixed Leukocyte Reaction (MLR) and Soluble Protein Presentation Assay

Autologous MLR was performed as described (Gruber, A., et al., (2001) J. Biol. Chem. 276:47840). Briefly, 5×10³ immature DC were incubated with 2×10⁵ autologous peripheral blood leukocytes (PBL) for 3 days without antigen or with various concentrations of tetanus toxoid. [³H]-thymidine (0.037 Mbq [1 μCi] per well; DuPont NEN, Boston, Mass.) was added 18 h before harvest, and incorporation of [³H]-thymidine into the cells was quantified using a β-counter (1450 Microbeta Wallac; Perkin Elmer, Boston, Mass.).

Allogeneic MLR

To assess the antigen-presenting cell function of DC, mature DC at varying concentrations were co-incubated with 2×10⁵ allogeneic PBL in 96-well flat bottom tissue culture microplates (Becton Dickinson) for 5 days. [³H]-thymidine (0.037 Mbq [1 Ci] per well) was added 18 h before harvest, and incorporation of [³H]-thymidine into the cells was quantified using a β-counter.

Statistics

Statistical significance was determined by paired two-way t-test; a p value of less than 0.05 was considered significant.

B. Results

DC-SIGN-Based Selection of DC

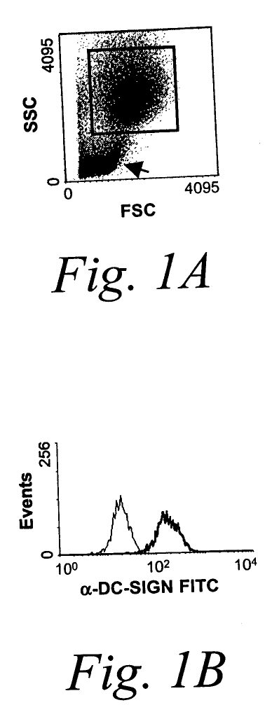

DC were generated from plastic-adherent PBMC in medium containing GM-CSF and IL-4. DC-SIGN was not expressed on plastic-adherent PBMC, but was rapidly upregulated (within 24 h) after addition of GM-CSF and IL-4. On day 5, the DC culture was incubated with FITC-conjugated antibody DCN46, and positively stained cells were collected by flow cytometry (FIG. 1). The majority of cells with high granularity (FIG. 1) stained positive for DCN46 (median: 95.9%; range: 69.8%-98.9%; n=5). The mean fluorescence intensity (MFI) of DC labeled with FITC conjugated antibody DCN46 differed markedly between experiments (median MFI: 68.6; range: 12.7-200.3; n=5). The latter finding may be due to different kinetics of DC-SIGN expression among the donors studied or to inter-individual variation in the expression of DC-SIGN (Mummidi, S., et al. (2001) J. Biol. Chem. 276:33196).

Detachment of DC-SIGN Antibody from DC Over Time

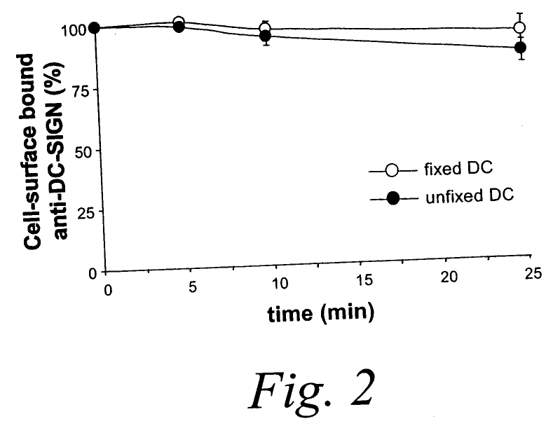

Recently, it has been shown that antibodies AZN-D1, AZN-D2, and AZN-D3 but not CSRD (all directed against DC-SIGN) are rapidly internalized from the cell surface (Engering, A., et al. (2002) J. Immunol. 168:2118). The monoclonal antibody DCN46 used in the present study was not rapidly internalized but remained on the cell surface of DC for a sufficiently long time to allow positive selection (FIG. 2). However, after incubation of previously antibody DCN46 stained DC for 2 days in culture medium at 37° C., no surface-bound DC-SIGN antibody was detectable by FACS. Furthermore, on day 2 after DC-SIGN-based DC enrichment, the mean fluorescence intensity of DC staining positive for DCN46 were similar between unsorted DC (MFI: 203±35; n=3; shown: mean±standard deviations) and sorted DC (MFI: 214±46; n=3). The latter findings show that DC bound DC-SIGN antibody was released or degraded after incubation of sorted DC for 2 days in culture medium, and that binding of antibody DCN46 to DC did not affect the expression level of DC-SIGN at this time point.

Immunophenotype of DC-SIGN-Sorted Immature DC

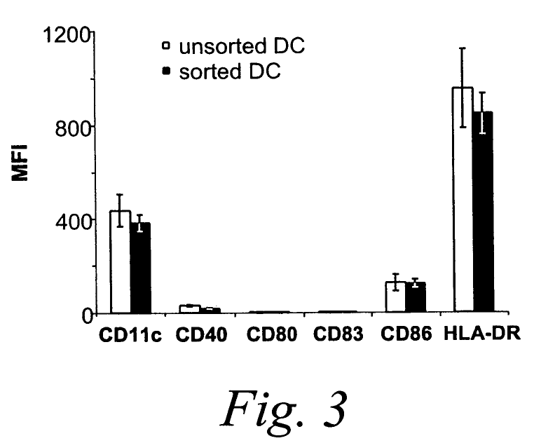

The majority of DC-SIGN-sorted DC cultured for 7 days with GM-CSF and IL-4 stained positive for HLA-DR, CD11c, CD86, and, at lower relative intensities, for CD40, CD80, and CD83 (FIG. 3). This profile is characteristic of functionally immature DC (Gruber, A., et al. (2000) Blood 96:1327; Pickl, W. F., et al. (1996) J. Immunol. 157:3850). The DC-SIGN-sorted DC population stained negative for CD3, CD14, CD19 and CD56; based on staining for HLA-DR, CD86, and CD11c, the DC-SIGN-sorted DC had a median purity of 99.4% (range: 97.9%-99.9%; n=5). In contrast, the purity of unsorted DC cultures was markedly lower (median: 69%; range: 30%-70%). The surface antigen expression profile of unsorted DC and DC-SIGN-sorted DC was comparable (FIG. 3). These findings suggest that binding of antibody DCN46 to DC does not affect the immunophenotype of immature DC.

DC-SIGN Sorting Does Not Affect the Ability of Immature DC to Take Up and Present Antigen

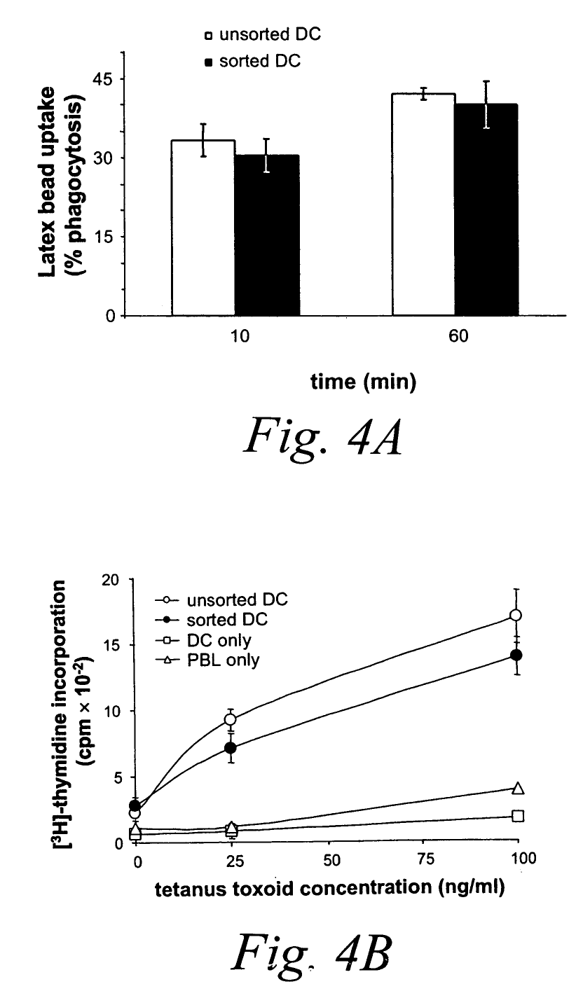

Phagocytosis of large particles is characteristic of immature DC (FIG. 4A) (Hart, D. N. J. (1997) Blood 90:3245). DC-SIGN sorting did not have any effect on the ability of DC to phagocytose latex beads (FIG. 4A). Furthermore, the ability of DC to process and present the recall antigen tetanus toxoid to autologous T cells was examined. Unsorted DC stimulated tetanus antigen specific T-cell proliferation in a dose-dependent manner (FIG. 4B). A similar dose-response curve was found for DC-SIGN-sorted DC (FIG. 4B).

DC-SIGN-Sorted DC Differentiate into Mature DC with Strong T-cell Stimulatory Capacity

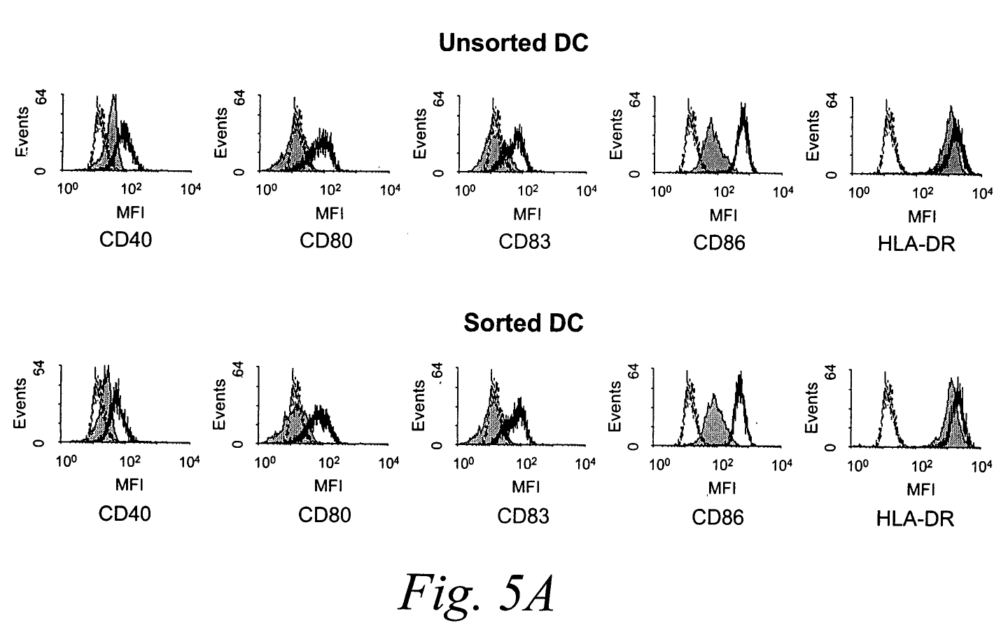

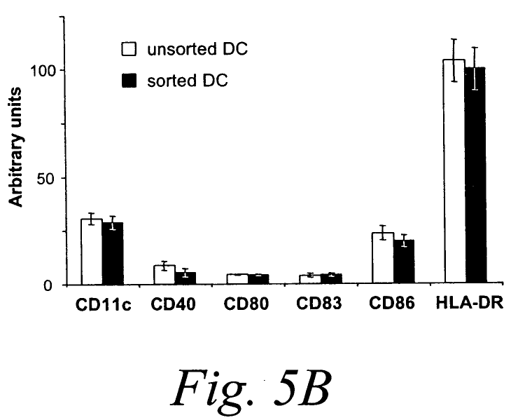

To investigate the effect of DC-SIGN sorting on DC maturation, immature DC were stimulated for 2 days with LPS. Maturation of DC was accompanied by a moderate to strong increase in expression of all surface antigens studied (FIG. 5A), except for CD11c which stayed unchanged. The effect of LPS on DC maturation varied between different donors, thus, to normalize data from experiments with DC from different donors, the mean fluorescence intensity of HLA-DR on DC-SIGN-sorted DC was arbitrarily set to 100 in each set of experiment (FIG. 5B). No difference in the expression level of the studied DC surface antigens was observed between unsorted and DC-SIGN-sorted DC (FIGS. 5A and 5B).

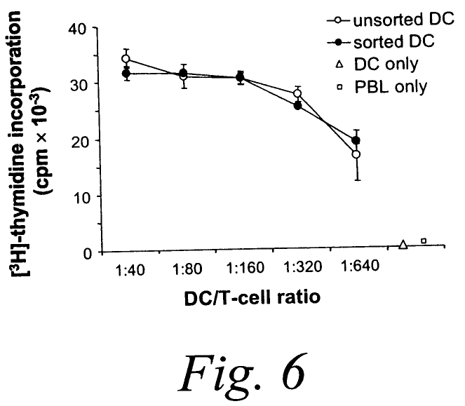

Recently, it has been shown that antibodies AZN-D1 and AZN-D2 directed against DC-SIGN inhibit DC-induced proliferation of allogeneic T cells (Geijtenbeek, T. B., et al. (2000) Cell 100:575). However, antibody DCN46-treated DC had the same capacity as unsorted DC to stimulate allogeneic T-cell proliferation (FIG. 6). At the time when the allogeneic mixed leukocyte reaction was performed, no DCN46 was detected on the surface of previously DC-SIGN-sorted DC.

### Equivalents

Those skilled in the art will recognize, or be able to ascertain using no more than routine experimentation, many equivalents to the specific embodiments of the invention described herein. Such equivalents are intended to be encompassed by the following claims.

1. A method of selecting dendritic cells from a heterogeneous population of cells comprising contacting said cells with an antibody against dendritic cell-specific adhesion receptor (DC-SIGN), or a fragment thereof, and identifying dendritic cells based on binding of said antibody to said cells, thereby selecting dendritic cells from a heterogeneous population of cells.

2. A method of selecting dendritic cells from a heterogeneous population of cells comprising the steps of:
(a) obtaining a sample comprising a heterogeneous population of cells;
(b) contacting said sample with an antibody against dendritic cell-specific adhesion receptor (DC-SIGN), or a fragment thereof;
(c) sorting said heterogeneous population of cells; and
(d) identifying cells that bind to said antibody, thereby identifying said dendritic cells.

3. A method of modulating an immune response in a subject comprising the steps of:
(a) obtaining a sample consisting of a heterogeneous population of cells;
(b) contacting said sample with an antibody against dendritic cell-specific adhesion receptor (DC-SIGN), or a fragment thereof,
(c) sorting said heterogeneous population of cells;
(d) identifying dendritic cells that bind to said antibody;
(e) contacting said dendritic cells with an antigen to produce antigenic cells; and
(f) administering said antigenic cells to said subject, thereby modulating an immune response in a subject.

4. A method of treating or preventing an immune disease or disorder in a subject comprising the steps of:
(a) obtaining a sample consisting of a heterogeneous population of cells;
(b) contacting said sample with an antibody against dendritic cell-specific adhesion receptor (DC-SIGN), or a fragment thereof;
(c) sorting said heterogeneous population of cells;
(d) identifying dendritic cells that bind to said antibody;
(e) contacting said dendritic cells with an antigen to produce antigenic cells; and
(f) administering said antigenic cells to said subject, thereby treating an immune disease or disorder in a subject.

5. The method of claim 3, wherein said immune response is an immune response to a disease or disorder selected from the group consisting of viral infection, bacterial infection, parasitic infection, prion disease, neoplastic disease, allergy, and autoimmunity.

6. The method of claim 4, wherein said immune disorder is selected from the group consisting of bacterial diseases, viral diseases, parasitic diseases, autoimmune diseases, allergy, and neoplastic diseases.

7. The method of either claims 3 or 4, wherein said antigen is derived from the group consisting of tumor cells, autoimmune cells, prions, bacterium, viruses, yeast and parasites.

8. The method of either claims 3 or 4, wherein said antigen is derived by recombinant means.

9. The method of either claims 3 or 4, wherein said subject is a mammal.

10. The method of claim 9, wherein said mammal is human.

11. A method of producing a vaccine comprising the steps of:
(a) obtaining a sample consisting of a heterogeneous population of cells;
(b) contacting said sample with an antibody against dendritic cell-specific adhesion receptor (DC-SIGN), or a fragment thereof;
(c) sorting said heterogeneous population of cells;
(d) identifying dendritic cells that bind to said antibody; and
(e) contacting said dendritic cells with an antigen, thereby producing a vaccine.

12. A kit to select dendritic cells from a heterogeneous population of cells comprising:
(a) an antibody against dendritic cell-specific adhesion receptor (DC-SIGN), or a fragment thereof; and
(b) instructions for use.

13. The method of claim 1 wherein said antibody is DCN46.

14. The method of claim 2 wherein said cells are sorted by flow cytometry cell sorting.

15. The method of claim 2 wherein said cells are sorted by magnetic beads.

16. The method of claim 1 wherein said antibody is fluorescently labeled.

17. The method of any one of claim 1 wherein said antibody is labeled by magnetic beads.

18. The method of claim 1 wherein said heterogeneous population of cells is derived from blood.

19. The method of claim 1 wherein said heterogeneous population of cells is derived from lymph.

20. The method of claim 1 wherein said heterogeneous population of cells is derived from lymph nodes.

21. The method of claim 1 wherein said heterogeneous population of cells is derived from spleen.

22. The method of claim 1 wherein said identified dendritic cells are 99% pure.

23. The method of claim 1 wherein said identified dendritic cells are 99.5% pure.

24. The method of any one of claims 3 or 4, wherein said antigenic cells are administered in a pharmaceutically acceptable formulation.

25. The kit of claim 12, wherein said antibody is DCN46.

26. The kit of claim 12, wherein said antibody is fluorescently labeled.

27. The kit of claim 12, wherein said antibody is labeled by magnetic beads.

28. The kit of claim 12, wherein said heterogeneous population of cells is derived from blood.

29. The method of claim 12, wherein said heterogeneous population of cells is derived from lymph.

30. The kit of claim 12, wherein said heterogeneous population of cells is derived from lymph nodes.

31. The kit of claim 12, wherein said heterogeneous population of cells is derived from spleen.
